(19) United States
(12) Patent Application Publication
(10) Pub. No.: US 2026/0218247 A1
(43) Pub. Date: Jul. 30, 2026

(54) METHOD FOR TREATING GRAINS TO PRODUCE MATERIAL USEFUL FOR CHEMICALS AND BIOFUELS

(71) Applicant: SHELL USA, INC., Houston, TX (US)

(72) Inventors: Claudia Ines ISHIZAWA HIGUCHI, Houston, TX (US); Juben Nemchand CHHEDA, Houston, TX (US); Shrikant Survase, Houston, TX (US)

(21) Appl. No.: 19/141,395

(22) PCT Filed: Jan. 18, 2024
(86) PCT No.: PCT/US2024/011981

§ 371 Date: Jun. 20, 2025

Related U.S. Application Data
(60) Provisional application No. 63/480,998, filed on Jan. 23, 2023.

(30) Foreign Application Priority Data
Feb. 9, 2023 (EP) 23155748.9

(51) Int. Cl. C12P 7/10; C12P 7/46; C12P 7/52; C12P 7/54; C12P 7/56

(52) U.S. Cl. CPC C12P 7/10; C12P 7/46; C12P 7/52; C12P 7/54; C12P 7/56; C12P 2203/00

(57) ABSTRACT

A method for treating grains having starch and non-starch carbohydrates, wherein the starch is present in an amount of at least 10 wt. % based on the dry weight of the grain. The grain is contacted with a solution containing at least one α-hydroxysulfonic acid; and to react under acid hydrolysis conditions to produce a product that is suitable for producing chemicals and/or fuel.

### FIELD OF THE INVENTION

The invention relates to a process for treating renewable feedstocks for the production of material useful for the production of chemicals and fuels, and in particular to a process for treating grains for the production of chemicals and fuels.

### BACKGROUND OF THE INVENTION

Many efforts to partially or completely replace fossil-derived resources for sustainable production of chemicals and/or fuels have focused on the use of corn, sugar cane, biomass and other agricultural residues. For example, in a corn dry mill process, the entire grain kernel is ground (milled) into flour. The flour is then physically and chemically prepared for fermentation via cooking to produce a mash, which is hydrolysed to release sugars using enzymatic saccharification. The sugar mixture, with solids, is then fermented to produce ethanol. The fermented product is distilled to separate ethanol from the unconverted solids and process water or stillage that remains at the bottom of the distillation tank. In a typical process, the whole stillage is separated into thin stillage (liquid fraction) and a solid fraction known as wet distillers grain (WDG).

The solids contain primarily unfermented grain residues (protein, fibre, fat). The kernel fibre contains sugars in the form of unconverted polysaccharides such as cellulose, hemicellulose, and resistant starch.

A primary obstacle to the use of the fibre in WDG is an expensive pretreatment step to release these sugars and make the cellulose and other polysaccharides in the feedstock accessible to enzymatic hydrolysis. One of the leading candidates for such a pretreatment is dilute mineral acid hydrolysis (typically sulfuric or hydrochloric acid). The conditions of a successful pretreatment in dilute acid hydrolysis are determined by a combination of three factors—time, temperature, and acid concentration. Increased temperatures lead to loss of sugars to degradation products and increasing acid concentration (to lower the temperature) comes at the expense of the acid employed and neutralized salts in downstream equipment.

Efforts toward separating sulfuric acid and sugars using ion resin separation or hydrochloric acid and sugars via amine extraction and subsequent thermal regeneration of the acid have been described by Farone et al. (U.S. Pat. No. 5,820,687, 1998 Oct. 13) and Baniel et al. (WO2010/026572A1, 2009 Sep. 1). Both approaches are cumbersome and expensive.

To address these problems, Tetarenko et al. (U.S. Pat. No. 9,428,816B2, 2016 Aug. 30) provide a method of treating an ethanol production by-product by treating WDG or stillage with a solution of α-hydroxysulfonic acid to produce a fermentable sugar for producing biofuels or ethanol.

A challenge remains for treating grains so that more sugars from starch and fibre can be recovered in a single process.

### SUMMARY OF THE INVENTION

According to one aspect of the present invention, there is provided a method for treating grains to produce a feedstock for producing chemicals and/or fuels, the method comprising the steps of: providing a grain comprising starch and non-starch carbohydrates, wherein the starch is present in an amount of at least 10 wt. % based on the dry weight of the grain; contacting the grain with a solution containing at least one α-hydroxysulfonic acid; and reacting the grain with the solution under acid hydrolysis conditions, thereby producing a feedstock suitable for producing chemicals and/or fuel.

### BRIEF DESCRIPTION OF THE DRAWINGS

The process of the present invention will be better understood by referring to the following detailed description of preferred embodiments and the drawings referenced therein, in which:

FIGS. 1 and 2 are block flow diagrams illustrating embodiments of the acid hydrolysis steps according to the method of the present invention;

FIG. 3 is a block flow diagram illustrating one embodiment of further processing of the product of the present invention by fermentation;

FIG. 4 is a block flow diagram illustrating another embodiment of further processing the product of the present invention by enzymatic hydrolysis and fermentation; and

FIG. 5 is a block flow diagram illustrating yet another embodiment of further processing the product of the present invention by catalytic conversion and/or other alternatives to fermentation.

### DETAILED DESCRIPTION OF THE INVENTION

The present invention provides a method for treating grain to produce a feedstock suitable for producing chemicals and fuels. The method allows for the treatment of grain without first requiring conversion, for example, by enzyme conversion, of starch to sugars. Advantageously, the grain may be processed in accordance with the method of the present invention without the need for energy-intensive grinding steps.

The method of the present invention allows the starch and non-starch carbohydrates in the grains to become more accessible to subsequent utilization, for example, via acid hydrolysis, with or without enzymatic hydrolysis, to produce sugars for further conversion to chemicals and/or fuels.

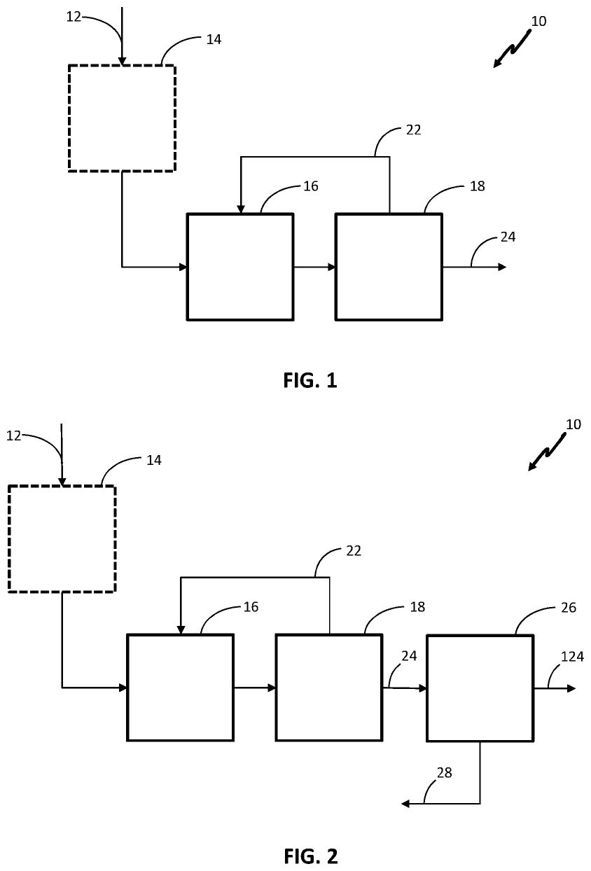

Referring now to FIG. 1, the method of the present invention 10 is provided to treat grains 12.

By “grains,” we mean cereal grains, grain legumes, oilseed grains, and combinations thereof. Examples of suitable cereal grains include, without limitation, wheat, rye, oats, barley, buckwheat, quinoa, chia, and combinations thereof. Examples of suitable grain legumes include, without limitation, beans, lentils, lupins, peas, peanuts, and combinations thereof. Examples of suitable oilseed grains include, without limitation, canola, corn, cottonseed, soy, sunflower, safflower, grapeseed, niger, castor, linseed, camelina, and combinations thereof. Preferably, the whole cereal grain, grain legume, and/or oilseed legume is used as a feed to the process of the present invention. In certain cases, the shells or husks may be used alone or together with the as a feedstock, cereal grains, grain legumes, oilseed grains, provided that the feedstock has the suitable properties as follows.

Suitable grains 12 have starch and non-starch carbohydrates. Starch is present in an amount of at least 10 wt. % based on the dry weight of the grain. Grains 12 also include non-starch carbohydrates including, without limitation, saccharides, lignin, cellulose, hemicellulose, glycogen, chitin, and combinations thereof. The starch content may be readily determined by those of ordinary skill in the art. Examples of methods for measuring starch content include, without limitation, polarimetry, HPLC, and the like.

Contrary to conventional methods where grains are pre-processed by to convert starch to sugar before acid hydrolysis, grains 12 may be treated in accordance with the method of the present invention without pre-processing. Alternatively, the grains 12 can be pre-processed to a suitable particle size that may include crushing and/or grinding. Suitable particle sizes will be dependent on the particular grain used. In a preferred embodiment, the grains 12 are subjected to an optional particle size reduction step 14. Advantageously, however, the method of the present invention does not require the grain to be subjected to an energy-intensive grinding step, for example, as required in conventional processes where grain is first ground to a flour before cooking.

An advantage of the optional particle size reduction step 14 is to open the grain for improved access to starch and non-starch carbohydrates in the subsequent treatment steps. The particle size reduction step 14 may simply be a step to crack open a shell or husk of the grain 12. In another embodiment, the particle size reduction step 14 may cause the grain to be split into two or more pieces. Suitable equipment for particle size reduction includes, for example, without limitation, hammer mills, jet mills, grinders, crushers, pulverisers, knife mills, shredders, and the like.

The grains 12 are passed to acid hydrolysis step 16, either directly or indirectly through optional particle size reduction step 14. In the acid hydrolysis step 16, grains 12 are contacted with a solution containing at least one α-hydroxysulfonic acid under acid hydrolysis conditions.

Suitable α-hydroxysulfonic acids for the method of the present invention have the general formula:

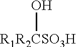

where R₁ and R₂ are independently hydrogen, an alkyl group having from 1 to 9 carbon atoms, or a hydroxyl group. The α-hydroxysulfonic acid can be a mixture of the acids having the general formula above.

The α-hydroxysulfonic acid can generally be prepared by reacting at least one carbonyl compound or a precursor of a carbonyl compound (e.g., trioxane, paraformaldehyde, metaldehyde, etc.) with sulphur dioxide or a precursor of sulphur dioxide (e.g., sulphur and oxidant, or sulphur trioxide and recuing agent) and water according to the following general Reaction 1.

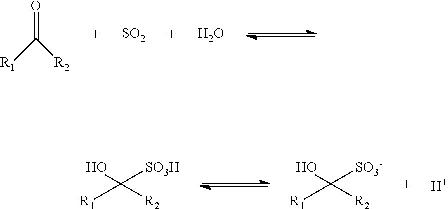

where R₁ and R₂ are independently hydrogen, an alkyl group with from 1 to 9 carbon atoms, or a hydroxyl group.

Illustrative examples of carbonyl compounds useful to prepare the α-hydroxysulfonic acids used in this invention are listed in Table 1:

TABLE 1

| Carbonyl Compound | R1 | R2 |
|---|---|---|
| Formaldehyde | H | H |
| Acetaldehyde | H | CH3 |
| Propionaldehyde | H | CH₂CH₃ |
| n-Butyraldehyde | H | CH₂CH₂CH₃ |
| i-Butyraldehyde | H | CH(CH₃)₂ |
| Glycolaldehyde | H | CH₂OH |
| Glyceraldehdye | H | CHOHCH₂OH |
| Glyoxal | H | C(═O)H |
| Furfural | H | C₄H₃O |
| Salicylaldehyde | H | C₆H₄OH |
| Benzaldehyde | H | C₆H₅ |
| Acetone | CH₃ | CH₃ |
| Acetol | CH₂OH | CH₃ |
| Methyl ethyl ketone | CH₃ | CH₂CH₃ |
| Mesityl oxide | CH₃ | CHC(CH₃)₂ |
| Methyl i-butyl ketone | CH₃ | CH₂CH(CH₃)₂ |
| Cyclohexanone | (CH₂)₅ | (CH₂)₅ |
| Chloroacetone | CH₃ | CH₂Cl |

The carbonyl compounds and/or a precursor thereof can be a mixture of compounds described above or a precursor thereof. For example, the mixture can be a carbonyl compound or a precursor such as, for example, trioxane which is known to thermally revert to formaldehyde at elevated temperatures or an alcohol that may be converted to an aldehyde by dehydrogenation of the alcohol by a method known to those skilled in the art. An example of such a conversion to aldehyde from alcohol is described below. An example of a source of carbonyl compounds may be a mixture of hydroxyacetaldehyde and other aldehydes and ketones produced from fast pyrolysis oil such as described in “Fast Pyrolysis and Bio-oil Upgrading, Biomass-to-Diesel Workshop”, Pacific Northwest National Laboratory, Richland, Washington, Sep. 5-6, 2006. The carbonyl compounds and its precursors can also be a mixture of ketones and/or aldehydes with or without alcohols that may be converted to ketones and/or aldehydes, preferably in a range of from 1 to 7 carbon atoms.

The preparation of α-hydroxysulfonic acids by the combination of an organic carbonyl compound, SO₂, and water is a general reaction and is illustrated below for acetone.

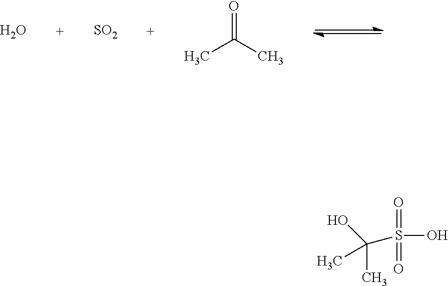

The α-hydroxysulfonic acids appear to be as strong as, if not stronger than, HCl.

In the acid hydrolysis step 16, the grain 12 is mixed with water and at least one α-hydroxysulfonic acid. The starch and non-starch polysaccharides in the grain 12 are converted into at least one fermentable sugar by the acid hydrolysis reaction with α-hydroxysulfonic acid.

The acid hydrolysis step 16 may comprise a number of components including α-hydroxysulfonic acid added or generated in situ. The term “in situ” as used herein refers to a component that is produced within the overall process; it is not limited to a particular reactor for production or use and is therefore synonymous with an in process generated component. When generated in situ, the carbonyl compound or incipient carbonyl compound (such as trioxane) should be added in an amount with sulphur dioxide and water, and under conditions effective to form α-hydroxysulfonic acids. The carbonyl compound or its precursor and sulphur dioxide or its precursor should be added in an amount to produce α-hydroxysulfonic acids (or recombined) in the range from about 1 wt. %, preferably from about 5 wt. %, most preferably from about 10 wt. %, to about 55 wt. %, preferably to about 50 wt. %, more preferably to about 40 wt. %, based on the total solution. For the reaction, excess sulphur dioxide is not necessary, but any excess sulphur dioxide may be used to drive the equilibrium in Reaction 1 to favour the acid form at elevated temperatures.

Various factors affect the conversion of the grains 12 in the acid hydrolysis step 16. The temperature and pressure of the acid hydrolysis step 16 should be in a range effective to form α-hydroxysulfonic acids and to hydrolyse grain starch and non-starch polysaccharides into hexoses and/or pentoses. The acid hydrolysis step 16 is conducted at temperature of at least 50° C. depending on the α-hydroxysulfonic acid used, although such temperature may be as low as room temperature (e.g., 20° C.) depending on the acid and the pressure used. The temperature of the acid hydrolysis step 16 may range preferably up to and including 150° C. depending on the α-hydroxysulfonic acid used. In a preferred embodiment, the temperature is in a range from 80° C. to 100° C. In a more preferred embodiment, the temperature is in a range from 90° C. to 130° C. The reaction is preferably conducted at as low a pressure as possible to contain the free sulphur dioxide used for generating α-hydroxysulfonic acids. The reaction may also be conducted at a pressure as low as 1 barg, preferably 4 barg, to a pressure as high as up to 10 barg. The selected temperature and pressure will depend on the particular α-hydroxysulfonic acid chosen and based on economic considerations of metallurgy and containment vessels, in a manner understood by those skilled in the art.

The amount of α-hydroxysulfonic acid solution to “dry weight” of the grain 12 determines the ultimate concentration of fermentable sugar obtained. Thus, as high a grain concentration as possible is desirable. This is balanced by the absorptive nature of grain with mixing, transport and heat transfer becoming increasingly difficult as the relative amount of grain solids to liquid is increased. The weight percentage of grains to total liquids may be in a range from 1 wt. % to 35 wt. % or even higher depending on the apparatus chosen and the nature of the grains 12.

The temperature and reaction time of the acid hydrolysis step 16 can be selected so that the maximum amount of extractable carbohydrates is hydrolysed and extracted as fermentable sugar from the grains 12 while limiting the formation of degradation products.

The acid hydrolysis step 16 may be conducted in a single reactor or a plurality of reactors. Suitable reactor designs include, for example, without limitation, batch, trickle bed, co-current, counter-current, stirred tank, and fluidized bed reactors. Staging of reactors can be employed to arrive the most economical solution. In one embodiment, a series of reactors may be used with an increasing temperature profile so that a desired sugar fraction is extracted in each reactor. The outlet of each reactor can then be cooled prior to combining the product streams 24, or the product streams 24 can be individually fed to subsequent steps.

The acid hydrolysis step 16 may be operated in a continuous, semi-continuous, batch, or semi-batch mode. Examples of continuous-flow systems include a continuous stirred-tank reactor (CSTR), a plug flow reactor, and combinations thereof. The acid hydrolysis reaction 16 may also be conducted in a multi-system vessel and/or reactor, in packed-bed flow-through reactors. In a preferred embodiment, the acid hydrolysis step 16 is practiced using a continuous-flow system operated at steady-state equilibrium.

The product stream 24 contains at least one fermentable sugar or monosaccharides, such as hexoses and/or pentoses, that are suitable for further processing.

After the acid hydrolysis step 16, the effluent is passed to an acid removal step 18. The reaction in Reaction 1 is a true equilibrium, which results in facile reversibility of the acid. That is, when heated, the equilibrium shifts towards the starting carbonyl compound, sulphur dioxide, and water. If the volatile components (e.g., sulphur dioxide) are allowed to depart the reaction mixture via vaporization or other methods, the acid reaction completely reverses, and the solution becomes effectively neutral. Thus, by increasing the temperature and/or lowering the pressure in the acid removal step 18, the sulphur dioxide can be driven off and the reaction completely reverses due to Le Chatelier's principle.

The fate of the carbonyl compound is dependent upon the nature of the material employed. If the carbonyl is also volatile (e.g., acetaldehyde), this material is also easily removed in the vapor phase. Carbonyl compounds such as benzaldehyde, which are sparingly soluble in water, can form a second organic phase and can therefore be separated by mechanical means. Thus, the carbonyl compound can be removed by conventional means, e.g., continued application of heat and/or vacuum, steam and nitrogen stripping, solvent washing, centrifugation, etc.

Therefore, the formation of the α-hydroxysulfonic acid is reversible in that as the temperature is raised, the sulphur dioxide and/or aldehyde and/or ketone can be flashed from the mixture and condensed or absorbed elsewhere in order to be recycled. It has been found that these reversible acids, which are approximately as strong as strong mineral acids, are effective in grains treatment reactions. We have found that these treatment reactions produce very few of the degradation products produced by other conventional mineral acids. Additionally, since the acids are effectively removed from the reaction mixture during the acid removal step 18, neutralization with base and the formation of salts to complicate downstream processing is substantially avoided. The ability to reverse and recycle these acids also allows the use of higher concentrations than would otherwise be economically or environmentally practical. As a direct result, the temperature employed in grains treatment can be reduced to diminish the formation of degradation products.

It has been found that the position of the equilibrium given in Reaction 1 at any given temperature and pressure is highly influenced by the nature of the carbonyl compound employed, steric and electronic effects having a strong influence on the thermal stability of the acid. More steric bulk around the carbonyl tending to favour a lower thermal stability of the acid form. Thus, one can tune the strength of the acid and the temperature of facile decomposition by the selection of the appropriate carbonyl compound.

The acid removal step 18 in the method of the present invention 10, in contrast to conventional mineral acid treatments, represents a particular advantage. Advantageously, the method of the invention results in potentially less expensive processing systems by eliminating subsequent acid neutralization steps and salt formation.

The effluent from the acid hydrolysis step 16 is introduced to acid removal step 18 where the acid is removed in its component form, then is recovered (and optionally scrubbed) either as components or in its recombined form and recycled via recycle stream 22 to acid hydrolysis step 16. Product stream 24, containing at least one fermentable sugar (e.g., hexoses and optionally pentoses), is substantially free of the α-hydroxysulfonic acid.

The acid removal step 18 may be conducted by applying heat and/or vacuum to reverse the formation of α-hydroxysulfonic acid to its starting material to produce a product stream 24 containing fermentable sugar substantially free of the α-hydroxysulfonic acid. In particular, the product stream 24 is substantially free of α-hydroxysulfonic acid, meaning no more than about 2 wt. % is present in the product stream 24, preferably no more than about 1 wt. %, more preferably no more than about 0.2 wt. %, most preferably no more than about 0.1 wt. % present in the product stream 24.

The temperature and pressure will depend on the particular α-hydroxysulfonic acid used. Preferably, the degree of heating is reduced to preserve the sugars obtained in the acid hydrolysis step 16. The acid removal 18 may be conducted at a temperature in a range from 50° C., preferably from 80° C., more preferably 90° C., to 150° C., preferably to 120° C. The pressure may be in a range of from 1 bara (atmospheric) to 3 bara, more preferably from 1 bara to 2 bara. It can be appreciated by a person skilled in the art that the acid hydrolysis step 16 and the acid removal step 18 can occur in the same vessel or independent vessels or in a number of different types of vessels depending on the reactor configuration and staging as long as the system is designed so that the reaction is conducted under condition favourable for the formation and maintenance of the α-hydroxysulfonic acid and removal favourable for the reverse reaction.

As an example, the reaction in the acid hydrolysis step 16 can be operated at a temperature of 100° C. and a pressure of 4 barg in the presence of α-hydroxyethane sulfonic acid, while the acid removal step 18 can be operated at 110° C. and a pressure of 0.5 barg. It is further contemplated that the reversion can be favoured by the reactive distillation of the formed α-hydroxysulfonic acid. In the recycle stream 22, optionally additional carbonyl compounds, SO₂, and water may be added as necessary. The removed starting material and/or α-hydroxysulfonic acid can be condensed or scrubbed by contact with water and recycled as a recycle stream 22 to the acid hydrolysis 16.

Turning now to FIG. 2, the product stream 24 may be used directly in the further processes or the product stream 24 may be passed to a solids separation step 26 for clarifying the product stream by removal of grain residue, such as protein.

The solids separation step 26 may include one or more solids separation equipment for purifying the liquid product stream by adsorption, distillation, crystallization, evaporation, precipitation, separation, or chemical reactions to remove impurities.

The solids separation step results in a solids stream 28 having a high protein content, preferably greater than 30 wt. % protein, more preferably greater than 40 wt. % protein, that may be further purified.

The product stream 24, 124 may be subjected to a number of different processes for further processing to chemicals and/or fuels, non-limiting examples of which are discussed further below. The product stream 24, 124 may be subjected to a process selected from biological, biochemical, chemical, and combinations thereof.

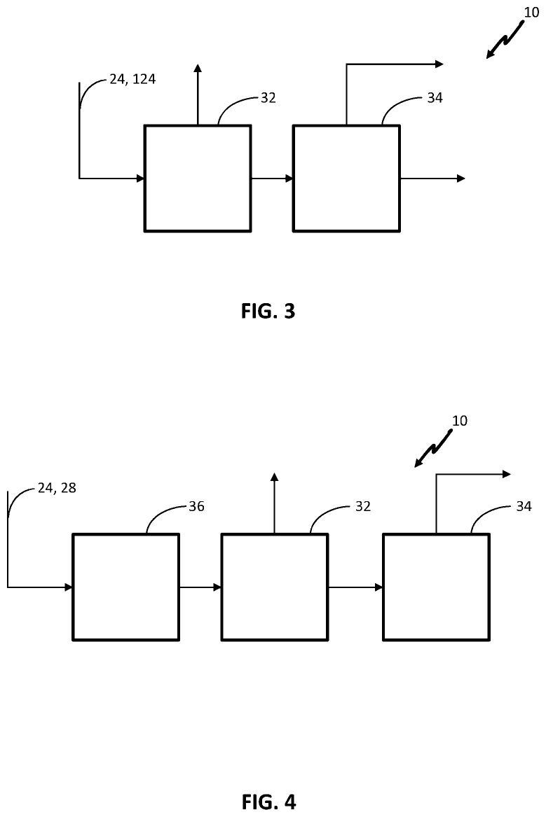

In one embodiment of a process for further processing, as illustrated in FIG. 3, the product stream 24, 124 is passed to a fermentation step 32 for conversion of the sugars to an alcohol useful, for example, as a biofuel.

In the fermentation step 32, one or more microorganisms (for example, yeasts or bacteria) may be used to convert sugar to biofuels, such as ethanol, other alcohol fermentation products, like n-butanol, iso-butanol, isopropanol, and/or organic acids, such as lactic acid, succinic acid, acetic acid, and propionic acid. The microorganisms convert sugars, including, but not limited to glucose, xylose, arabinose, mannose, and galactose present in the product stream 24, 124 to a fermentation product.

Suitable microorganisms include bacteria, yeast, and fungi. Examples of suitable bacteria include, without limitation, Clostridia, Escherichia coli (E. coli), recombinant strains of E. coli, genetically modified strains of Zymomonas mobilis (such as described in Zhang et al. (U.S. Pat. No. 7,223,575B2, 2007 May 29) and Viitanen et al. (U.S. Pat. No. 7,741,119B2, 2010 Jun. 22; U.S. Pat. No. 7,741,084B2 2010 Jun. 22)), and combinations thereof. Examples of suitable yeast or fungus include, without limitation, species of Saccharomyces, Kluyveromyces, Candida, Pichia, Schizosaccharomyces, Hansenula, Kloeckera, Schwanniomyces, Yarrowia, Aspergillus, Trichoderma, Humicola, Acremonium, Fusarium, Penicillium, and combinations thereof. The fermentation may also be performed with recombinant yeast engineered to ferment both hexose and pentose sugars to ethanol. Recombinant yeasts that can ferment one or both of the pentose sugars xylose and arabinose to ethanol are described in Ho et al. (U.S. Pat. No. 5,789,210 1998 Aug. 4), Otero et al. (U.S. Pat. No. 6,475,768B1 2002 Nov. 5), Gorwa-Grauslund et al. (EP1727890B1 2008 May 14), and Boles et al. (WO2006/096130A1 2006 Sep. 14). Xylose utilization can be mediated by the xylose reductase/xylitol dehydrogenase pathway (for example, Ho et al. (WO97/42307 1997 Nov. 13) or the xylose isomerase pathway (for example, Concilio et al. (WO2007/028811A1 2006 Sep. 6) and Klaassen et al. (WO2009/109631A1 2009 Sep. 11). It is also contemplated that the fermentation organism may also produce fatty alcohols, for example, as described in Hu et al. (WO2008/119082A2 2008 Oct. 2) and Keasling et al. (WO2007/136762A2 2007 May 18). In another embodiment, the fermentation may be performed by yeast capable of fermenting predominantly C6 sugars for example by using commercially available strains such as THERMOSACC® and SUPERSTART®.

Preferably, the fermentation step 32 is performed at or near the temperature and pH optima of the selected fermentation microorganism. For example, the temperature may be in a range from 25° C. to 55° C. The pH may be in a range of from 3.0 to 7.0. The dose of the fermentation microorganism will depend on a number of parameters, such as the activity of the fermentation microorganism, the desired fermentation time, the volume of the reactor, and the like. It will be appreciated that these parameters may be adjusted as desired by one of skill in the art to achieve optimal fermentation conditions.

The fermentation step 32 may be conducted in batch, continuous, or fed-batch modes, with or without agitation. The fermentation step 32 may employ a series of fermentation reactors (not shown).

The fermentation step 32 produces an overhead CO₂ stream and a liquid alcohol-containing stream, preferably containing at least one alcohol having from 2 to 18 carbon atoms. The liquid alcohol-containing stream is passed to a recovery step 34. The recovery step 34 may include one or more of distillation columns, solid/liquid separation apparatus, evaporators, centrifuges, decantation tanks, skimmers, filters, membranes, and the like.

When the product to be recovered in the alcohol-containing stream is a distillable alcohol, such as ethanol, the alcohol can be recovered from an aqueous stream by distillation in the recovery step 34. When the product to be recovered in the alcohol-containing stream is not a distillable alcohol, such as fatty alcohols, the alcohol can be recovered in the recovery step 34 by removal of alcohols as solids or as oils.

In one embodiment of the recovery step 34, the effluent from the fermentation step 34 is passed to a distillation column to separate ethanol from the effluent. The bottoms stream from the distillation column is then passed to a solid/liquid separation to separate a solid stream from a liquid stream. The liquid stream from the solid/liquid separation is passed to an evaporator to produce a concentrated liquid or syrup. The concentrated liquid or syrup is then passed to a centrifuge for removing oil from a de-oiled syrup. In one embodiment, oil may also be recovered from the evaporator. The de-oiled syrup is rich in protein, useful, for example, as an animal feed. The oil removed from the liquid stream is useful as a bio-oil feedstock for processing to fuels and/or chemicals.

In the embodiment of FIG. 1, where the product stream 24 is passed to the fermentation step without first passing through a solids separation step, the solids recovered by a solid/liquid separation from the effluent of the recovery step 34 will include unconverted solids from the acid hydrolysis 16 of the grains 12, as well as fermentations solids.

In the embodiment of FIG. 2, where the product stream 124 is passed to the fermentation step after passing through the solids separation step 26, the solids recovered by a solid/liquid separation in the recovery step 34 will include fermentations solids.

Referring now to FIG. 4, another embodiment of further processing provides that the product stream 24 and/or solids stream 28 is subjected to enzyme hydrolysis 36 thereby providing a hydrolysate.

The enzyme hydrolysis 36 and fermentation 32 steps may be conducted in separate vessels or in the same vessel. In one embodiment, the enzyme hydrolysis 36 can be partially completed, and the partially hydrolysed stream may be fermented in fermentation step 32. In another embodiment, simultaneous saccharification and fermentation (SSF) may be conducted, where enzyme hydrolysis 36 may be run until the final percent solids target is met and then the hydrolysed biomass may be fermented in fermentation step 32.

In the enzyme hydrolysis step 36, the product stream 24 and/or solids stream 28 is contacted with cellulase, β-glucosidase, xylanase, glucoamylase or combinations thereof. Depending on the temperature, the product stream 24 and/or solids stream 28 may be adjusted to a temperature within the optimum range for the activity of the enzymes. Generally, a temperature in a range of from 15° C. to 100° C., preferably from 20° C. to 85° C., more preferably from 30° C. to 70° C., is suitable for most cellulase enzymes. The temperature may be adjusted by heat exchange before being introduced to the enzyme hydrolysis step 36. Alternatively, the enzymes may be added to the product stream 24, prior to, during, or after the adjustment of the temperature and pH of the product stream 24 in the hydrolysis reaction vessel. Preferably the enzymes are contacted with the product stream 24 after temperature and pH have been adjusted. The desired operating conditions, including temperature and pH, will be determined depending on the selected enzyme.

Cellulase is an enzyme or a mixture of enzymes capable of hydrolysing cellulose. A cellulase enzyme mixture may include cellobiohydrolases (CBH), glucobiohydrolases (GBH), endoglucanases (EG), and β-glucosidase. The term “β-glucosidase” refers to enzymes that hydrolyse the glucose dimer, cellobiose, to glucose.

The enzymatic hydrolysis step 36 may also be carried out in the presence of one or more xylanase enzymes. Examples of xylanase enzymes include, for example, without limitation, xylanase 1, 2 (Xyn1 and Xyn2), and β-xylosidase.

Sources of cellulases include, for example, without limitation, species of Aspergillus, Humicola, Trichoderma, Myceliophthora, Chrysosporium, Bacillus, Thermobifida, Thermotoga, Acremonium, Aureobasidium, Bjerkandera, Ceriporiopsis, Coprinus, Coriolus, Cryptococcus, Filibasidium, Fusarium, Magnaporthe, Mucor, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and combinations thereof.

The cellulase enzyme dosage is selected to convert the cellulose in the product stream 24 and/or solids stream 28 to glucose. For example, an appropriate cellulase dosage can be in a range from 10 to 150 Biomass Hydrolysis Unit(s) (BHU) per gram of cellulose. The term Biomass Hydrolysis Unit(s) refers to the enzyme concentration.

In practice, the enzyme hydrolysis 36 may carried out in one or a series of hydrolysis reactors. The number of hydrolysis reactors in the system depends on the cost of the reactors, the volume of the aqueous slurry, and other factors. The enzymatic hydrolysis 36 with cellulase enzymes produces an aqueous sugar stream (hydrolysate) comprising glucose, unconverted cellulose, lignin and other sugar components. The hydrolysis may be carried out in two stages (see, for example, Brink (U.S. Pat. No. 5,536,325 1996 Jul. 16)) or may be performed in a single stage.

In SSF, a combination of enzyme and yeast are used to complete the conversion of polysaccharides to sugars and simultaneously fermenting the sugars to ethanol and carbon dioxide, producing fermented products containing ethanol, solids from the grain (in the case where solids are not first separated in the embodiment of FIG. 2), water, and carbon dioxide that can be removed or captured.

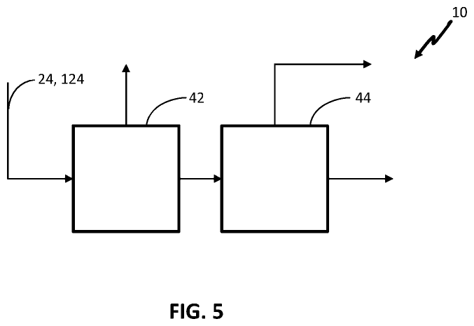

Referring now to FIG. 5, another embodiment of further processing provides that the product stream 24, 124 is subjected to catalytic hydrogenation and condensation. In this embodiment, the product stream 24, 124 is subjected to a hydrogenolysis step 42 where it is contacted with hydrogen in the presence of a hydrogenolysis catalyst to form a plurality of oxygenated intermediates. The oxygenated intermediates are then further processed in condensation step 44 to generate a fuel blend catalysed by a catalyst comprising acid and/or basic functional sites, to produce a liquid fuel. In this way, the product stream 24, 124 is converted to higher hydrocarbons useful as a biofuel component. As used herein, the term “higher hydrocarbons” refers to hydrocarbons having an oxygen to carbon ratio less than at least one component of the grain feedstock.

In one such example, the product stream 24, 124 is further processed to produce mixtures of C4+ compounds useful for biofuels such as described in Chheda et al. (US2011/0154721A1 2011 Jun. 30 and US2011/0282115A1 2011 Nov. 17), which disclosures are hereby incorporated by reference. As another such example, the product stream 24, 124 may be further processed to produce mixtures of C4+ compounds useful for biofuels such as described in Cortright et al. (US2008/0216391A1 2008 Sep. 11), which disclosure is hereby incorporated by reference. The solids stream 28 may also be suitable for use in fast pyrolysis reactions leading to fuels and chemicals.

While the invention is susceptible to various modifications and alternative forms, specific embodiments thereof are shown by way of examples herein described in detail. It should be understood that the detailed description herein is not intended to limit the invention to the particular form disclosed, but on the contrary, the intention is to cover all modifications, equivalents and alternatives falling within the spirit and scope of the present invention as defined by the appended claims.

### EXAMPLES

The following non-limiting examples of embodiments of the method of the present invention as claimed herein are provided for illustrative purposes only.

### General Methods and Materials

In the examples, the aldehyde or aldehyde precursors and sulphur dioxide were obtained from Sigma-Aldrich Co.

Corn kernels and sorghum grains were obtained via commercial sources. The composition of the material was analysed using standard NREL methods (TP-510-48087) and the results following average composition on a dry basis are presented in Table 2:

TABLE 2

| | Corn | Sorghum |
|---|---|---|
| | (wt. %) | (wt. %) |
| Glucan (starch and cellulose) | 76.44 | 76.58 |
| Xylan | 2.52 | 1.45 |
| Arabinan | 2.09 | 1.83 |
| Lignin | 3.58 | 3.98 |
| Ash | 0.95 | 1.16 |
| Protein | 8.50 | 8.59 |
| Hexane Extractives | 2.36 | 2.29 |

### Analytical Methods

### Determination of Oxygenated Components in Aqueous Layer.

A sample or standard was analysed by injection into a stream of a mobile phase that flowed through a Bio-rad column (Aminex HPX-87H, 300 mm×7.8 mm). The reverse phase HPLC system (Shimadzu) was equipped with both RI and UV detectors and the signals were recorded as peaks on a data acquisition and data processing system. The components were quantified using external calibration via a calibration curve based on injection of known concentrations of the target components. Some of the components were calculated by using single point of standard.

### HPLC Instrument Conditions:

Column: Bio-Rad Aminex HPX-87H (300 mm×7.8 mm)
Flow Rate: 0.6 ml/minute
Column Oven: 30° C.
Injection Volume: 10 μl
UV Detector: @320 NM
RI Detector: mode—A; range—100
Run Time: 70 minutes
Mobile Phase: 5 mM Sulfuric Acid in water

The sample was either injected directly or diluted with water first but without particulates. If there was precipitation in the sample or diluted sample, the sample was passed through a 0.2 μm syringe filter. Samples were analysed for Glucose, Xylose, Arabinose, Acetic Acid, Hydroxymethyl furfural, and Furfural content.

### General Procedure for the Formation of α-Hydroxysulfonic Acids

Aldehydes and ketones will readily react with sulphur dioxide in water to form α-hydroxysulfonic acids according to the Reaction 1 above. These reactions are generally rapid and somewhat exothermic. The order of addition (SO₂ to carbonyl or carbonyl to SO₂) did not seem to affect the outcome of the reaction. If the carbonyl compound was capable of aldol reactions, preparation of concentrated mixtures (>30 wt. %) was conducted at temperatures below ambient to minimize side reactions. The course of the reaction was tracked using in situ Infrared Spectroscopy (ISIR) employing probes inserted into pressure reaction vessels or systems (Mettler Toledo Autochem's Sentinal probe). In addition to being able to see the starting materials: water (1640 cm⁻¹), carbonyl (from approx. 1750 cm⁻¹ to 1650 cm⁻¹ depending on the organic carbonyl structure) and SO₂ (1331 cm⁻¹), the formation of the α-hydroxysulfonic acid is accompanied by the formation of characteristic bands of the SO₃⁻ group (broad band around 1200 cm⁻¹) and the stretches of the α-hydroxy group (single to multiple bands around 1125 cm⁻¹). In addition to monitoring the formation of the α-hydroxy sulfonic acid, the relative position of the equilibrium at any temperature and pressure can be readily assessed by the relative peak heights of the starting components and the acid complex. The definitive presence of the α-hydroxysulfonic acid under biomass hydrolysis conditions can also be confirmed with the ISIR and it is possible to monitor the growth of sugars in the reaction mixture by monitoring the appropriate IR bands.

### Example 1

Formation of 40 wt. % α-Hydroxyethane Sulfonic Acid from Acetaldehyde.

Into a 2-liter C-276 Parr autoclave fitted with DiComp IR optics was added 1346.1 grams of ice-cold nitrogen degassed aqueous solution containing 17.96 wt. % of acetaldehyde. The top was place on the reactor and the vessel was connected to two single ended Hoke vessels charged with a total of 362.04 grams of sulfur dioxide. The sealed reactors pressure integrity was ensured by a 15-minute test at 50 psig with nitrogen gas. The reactor was cooled to less than 5° C. using external chilling and the nitrogen cap was vented. The IR acquisition was initiated followed several minutes later by injection of the sulfur dioxide from the attached Hoke vessels to the acetaldehyde/water solution. The pressure in the reactor spiked to approximately 3 bar and then rapidly dropped to atmospheric pressure as the ISIR indicated the appearance and then rapid consumption of the SO₂. The temperature of the reaction mixture rose approximately 42° C. during the formation of the acid (from 3° C. to 45° C.). ISIR and reaction pressure indicated the reaction was complete in approximately 10 minutes. The final solution showed an infrared spectrum with the following characteristics: a broad band centred about 1175 cm⁻¹ and two sharp bands at 1038 cm⁻¹ and 1015 cm⁻¹. The reaction mixture was cooled to room temperature and the atmosphere contained vented through a caustic scrubber. The reactor was purged to remove any unreacted SO₂ or acetaldehyde by two cycles of pressurization with nitrogen to 50 psig and then venting. This produced 1698.78 g of a mixture that was analysed via proton NMR to contain 39.3 wt. % α-hydroxyethane sulfonic acid.

### Example 2

Formation of 40 wt. % α-Hydroxyethane Sulfonic Acid from Metaldehyde

Into a 2-litre C-276 Parr autoclave fitted with DiComp IR optics was added 999.98 grams of nitrogen degassed water and 212.02 g of metaldehyde (water insoluble). The vessel head was attached and to this was connected to two single ended Hoke vessels charged with a total of 338.19 grams of sulphur dioxide. The reactor was sealed, and the pressure integrity was confirmed by a 15-minute test at 100 psig with nitrogen gas. The nitrogen cap was vented very slowly to prevent any loss of free metaldehyde not wetted by the water. Stirring was initiated at 1000 rpm and the IR acquisition was begun. After 5 minutes the sulphur dioxide from the attached Hoke vessels was added to the metaldehyde/water slurry. The pressure in the reactor spiked to approximately 2.5 bar. ISIR indicated the appearance of the SO₂. The temperature of the reaction mixture was slowly increased to 30° C. then incrementally by 10° C. to 50° C. During transition from 40 to 50° C. the reaction temperature abruptly rose to 65° C. and the ISIR indicated formation of the acid. This was accompanied by the consumption of SO₂ and a fall in the reactor pressure. ISIR of the reaction mixture indicated the reaction was complete within a few minutes. The final solution showed an infrared spectrum with the following characteristics: a broad band centred about 1175 cm⁻¹ and two sharp bands at 1038 cm⁻¹ and 1015 cm⁻¹. The reaction mixture was cooled to room temperature and the atmosphere contained vented through a caustic scrubber. The reactor was purged to remove any unreacted SO₂ or acetaldehyde by two cycles of pressurization with nitrogen to 50 psig and then venting. This produced 1468.74 g of a light-yellow homogeneous liquid that was analysed via proton NMR to contain 36.7 wt. % α-hydroxyethane sulfonic acid.

### Examples 3-8

Hydrolysis of Corn Kernels and Sorghum Grains with α-Hydroxyethane Sulfonic Acid (HESA) Solutions

This is the general procedure for Examples 3 through 8. Into a 1000 ml autoclave equipped with a DiComp IR probe place approximately 100 to 150 grams of corn kernels (Examples 3-5) or sorghum grains (Examples 6-8) as obtained (dry wt. basis is recorded in column B). To this approximately 350 to 400 grams of a 4.5 wt. % α-hydroxyethane sulfonic acid solution (exact concentration listed in column C, Table 3) at room temperature.

The reactor was sealed with a top containing a magnetically coupled 4 blade-down pitch impeller, fitted with heating bands and purged by adding nitrogen to 50 psig followed by venting to room temperature. The reaction mixture was heated to the target temperature of 115 to 125° C. (column D) with stirring (1000 rpm) and held at temperature the requisite time (column E).

During this period of time, the in-situ IR reveals the presence of the acid, SO₂, and acetaldehyde in an equilibrium mixture and a growth in bands characteristic of the sugars around 1000 cm⁻¹. At the end of the reaction period, the acid reversal was accomplished via opening the gas cap of the reactor to an overhead condensation system for recovery of the acid while maintaining the reactor temperature at 100° C. This overhead recovery system was a ¼″ C-267 tube that extends downward into a 500 ml three neck round bottom flask charged with 150 grams of DI water, immersed in a wet ice bath and fitted with a dry ice/acetone condenser on the outlet.

The progress of the acid reversion was monitored via the use of in situ IR in the reactor. The reversal was continued until the in-situ IR showed no remaining traces of the α-hydroxyethane sulfonic acid or SO₂ in the reaction mixture. The reactor condensation system was then closed and the reactor cooled to room temperature. The overhead condensate contained >70% of the α-hydroxyethane sulfonic acid charged to the system as analysed by proton NMR in all cases (column F). The cooled reactor was opened and the contents filtered through a medium glass frit funnel using a vacuum aspirator to draw the liquid through the funnel. The reactor was rinsed with three separate portions of water, noting weight on all rinses, rinses being used to complete the transfer of solids and rinse the solids in the funnel. The residual solid was dried to a constant weight in the air and then analysed for moisture (the recovered weight on a dry basis in listed in column G). HPLC analysis of the filtrate plus rinses was used to obtain the results of hydrolysis (columns H to J).

TABLE 3

| | B | | | | | G | | | | |
|---|---|---|---|---|---|---|---|---|---|---|
| | Grain | C | D | E | F | Solids | H | I | J | K |
| | Dry | Acid | Reaction | Reaction | Acid | Recovered | Glucose | Xylose | Arabinose | Furfural |
| A | wt. basis | Conc. | Temp. | Time | Recovered | (g) dry wt. | (g) in | (g) in | (g) in | (g) in |
| Ex. | (g) | (wt. %) | (° C.) | (hr) | (wt. %) | basis | filtrate | filtrate | filtrate | filtrate |
| 3 | 150 | 4.7 | 120 | 1.0 | 78.6 | 5.90 | 90.2 | 5.4 | 2.1 | 0.18 |
| 4 | 100 | 4.2 | 120 | 1.0 | 76.2 | 2.42 | 65.1 | 3.0 | 1.7 | 0.16 |
| 5 | 100 | 4.2 | 120 | 0.5 | 75.6 | 2.49 | 63.8 | 3.1 | 1.6 | 0.11 |
| 6 | 150 | 4.7 | 120 | 1.0 | 76.2 | 5.93 | 95.0 | 3.5 | 1.9 | 0.18 |
| 7 | 100 | 4.2 | 120 | 1.0 | 74.7 | 3.37 | 68.5 | 1.9 | 1.3 | 0.14 |
| 8 | 100 | 4.2 | 120 | 0.5 | 73.1 | 3.57 | 68.8 | 1.9 | 1.3 | 0.07 |

The examples demonstrate that more than 75% of the glucans in corn kernels and sorghum grain, either from starch or cellulose, were converted into glucose with the remaining glucans as soluble oligomers and little sugar degradation as indicated by the low furfural concentration in the filtrate. Furthermore, the sugar stream was fermented to produce ethanol at same yield level as sugar streams from dry corn ethanol plants and more than 90% of the monomeric sugars consumed during fermentation.

### Supplementary Evidence

Based on the results presented in Blackbourn et al. (U.S. Pat. No. 9,290,821B2 22 Mar. 2016), the present inventors expect that similar results to those presented herein for α-hydroxyethane sulfonic acid will be achieved for other α-hydroxysulfonic acids. Specifically, Table 1 of Blackbourn et al., repeated below, shows that xylose, glucose, and furfural recovery can be comparable for α-hydroxyethane sulfonic acid, α-hydroxymethane sulfonic acid, and bis-α-dihydroxymethane sulfonic acid. Although Blackbourn et al. uses a different starting material (namely, biomass and WDG) than the invention herein, it is expected that the relative effectiveness of other α-hydroxysulfonic acids would be reasonably expected in the present method for grains having starch and non-starch carbohydrates.

TABLE 4
(copied from Blackbourn et al.)

| | | C | D | E | F | G | H | |
|---|---|---|---|---|---|---|---|---|
| | | Acid | Reaction | Time at | % Xylose | % Glucose | % Furfural | I |
| A | B | Concentration | Temperature | Temperature | Recovery in | Recovery | Recovery | Stirrer |
| Example | Acid | (% Wt) | (° C.) | (hr) | Filtrate* | in Filtrate** | in Filtrate* | Speed |
| 6 | α-hydroxyethane | 10 | 80 | 22.5 | 55.43 | 3.16 | 0.37 | 1000 |
| | sulfonic acid | | | | | | | rpm |
| 7 | α-hydroxyethane | 10 | 80 | 24 | 55.26 | 4.24 | 0.51 | 1000 |
| | sulfonic acid | | | | | | | rpm |
| 8 | α-hydroxyethane | 40 | 80 | 5 | 55.97 | 3.84 | 0.60 | 1000 |
| | sulfonic acid | | | | | | | rpm |
| 9 | α-hydroxyethane | <1 | 80 | 26.5 | 15.09 | 0.59 | ND | 1000 |
| | sulfonic acid | | | | | | | rpm |
| 10 | α-hydroxyethane | 10 | 80 | 0.1 *** | 2.56 | 0.41 | ND | 1000 |
| | sulfonic acid | | | | | | | rpm |
| 11 | α-hydroxyethane | 10 | 80 | 3.0 | 54.89 | 1.55 | ND | 1000 |
| | sulfonic acid | | | | | | | rpm |
| 12 | α-hydroxyethane | 10 | 100 | 0.1 *** | 46.17 | 1.28 | 0.073 | 1000 |
| | sulfonic acid | | | | | | | rpm |
| 13 | α-hydroxyethane | 10 | 90 | 2 | 66.16 | 3.16 | 0.47 | 1000 |
| | sulfonic acid | | | | | | | rpm |
| 14 | α-hydroxyethane | 10 | 100 | 1 | 65.02 | 4.32 | 0.27 | 1000 |
| | sulfonic acid | | | | | | | rpm |
| 15 | α-hydroxyethane | 10 | 100 | 2 | 75.60 | 6.51 | 1.63 | 1000 |
| | sulfonic acid | | | | | | | rpm |
| 16 | α-hydroxyethane | 10 | 100 | 4 | 82.7 | 7.79 | 3.33 | 1000 |
| | sulfonic acid | | | | | | | rpm |
| 17 | α-hydroxyethane | 10 | 90 | 1 | 66.12 | 3.77 | 0.54 | 1500 |
| | sulfonic acid | | | | | | | rpm |
| 18 | α-hydroxyethane | 20 | 90 | 2 | 72.9 | 5.65 | 1.36 | 1500 |
| | sulfonic acid | | | | | | | rpm |
| 19 | α-hydroxyethane | 10 | 90 | 4 | 70.68 | 5.50 | 1.70 | 1500 |
| | sulfonic acid | | | | | | | rpm |
| 20 | α-hydroxyethane | 20 | 100 | 1 | 73.46 | 6.20 | 1.02 | 1500 |
| | sulfonic acid | | | | | | | rpm |
| 21 | α-hydroxymethane | 10 | 80 | 22 | 44.46 | 4.14 | 0.67 | 1000 |
| | sulfonic acid | | | | | | | rpm |
| 22 | α-hydroxymethane | 10.25 | 120 | 3.3 | 55.48 | 12.58 | 9.37 | 1000 |
| | sulfonic acid | | | | | | | rpm |
| 23 | α-hydroxymethane | 41 | 80 | 25.5 | 15.33 | 12.43 | 0.82 | 1000 |
| | sulfonic acid | | | | | | | rpm |
| 24 | α-hydroxymethane | 41 | 80 | 0.1 *** | 67.12 | 4.66 | 0.88 | 1000 |
| | sulfonic acid | | | | | | | rpm |
| 25 | α-hydroxymethane | 10 | 80 | 0.1 *** | 8.61 | 0.48 | ND | 1000 |
| | sulfonic acid | | | | | | | rpm |
| 26 | Bis-α- | 10.6 | 80 | 2 | 53.85 | 1.57 | ND | 1000 |
| | dihydroxymethane | | | | | | | rpm |
| | sulfonic acid | | | | | | | |
| 27 | Bis-α- | 10.6 | 80 | 21 | 73.16 | 5.42 | ND | 1000 |
| | dihydroxymethane | | | | | | | rpm |
| | sulfonic acid | | | | | | | |

*Based on % w xylan in the feed (0.88 grams of xylan produces 1.0 grams of xylose theoretical)
**Based on % w glucan in the feed
*** Reactor was brought to the designated temperature and shutdown immediately

1. A method for treating grains to produce a feedstock for producing chemicals and/or fuels, the method comprising the steps of:
providing a grain comprising starch and non-starch carbohydrates, wherein the starch is present in an amount of at least 10 wt. % based on the dry weight of the grain;
contacting the grain with a solution containing at least one α-hydroxysulfonic acid; and
reacting the grain with the solution under acid hydrolysis conditions, thereby producing a feedstock suitable for producing chemicals and/or fuel.

2. The method of claim 1, wherein the grain is selected from the group consisting of cereal grains, grain legumes, oilseed grains, and combinations thereof.

3. The method of claim 1, further comprising the step of removing the α-hydroxysulfonic acid from the product by heating and/or reducing pressure to produce an acid-removed product containing at least one fermentable sugar substantially free of the α-hydroxysulfonic acid.

4. The method of claim 3, further comprising the step of recycling the removed α-hydroxysulfonic acid to the contacting step as components or in its recombined form.

5. The method of claim 3, further comprising the step of fermenting the acid-removed product to produce at least one alcohol.

6. The method of claim 3, further comprising the step of fermenting the acid-removed product to produce at least one organic acid.

7. The method of claim 3, further comprising the step of hydrolysing the acid-removed product.

8. The method of claim 7, further comprising fermenting the hydrolysed product to produce at least one alcohol.

9. The method of claim 1, wherein the solution has a concentration of α-hydroxysulfonic acid in a range of from 1 wt. % to 55 wt. %.

10. The method of claim 1, wherein the α-hydroxysulfonic acid is produced from a carbonyl compound or a precursor to a carbonyl compound with sulphur dioxide and water.

11. The method of claim 1, wherein the α-hydroxysulfonic acid is generated in-situ.

12. The method of claim 1, wherein the contacting step is carried out at a temperature in the range of about 50° C. to about 150° C. and a pressure in the range of 1 barg to about 10 barg.
